(19) United States
(12) Patent Application Publication
(10) Pub. No.: US 2002/0098176 A1
(43) Pub. Date: Jul. 25, 2002

(54) Inhibition of tumor growth by a nematode anticoagulant protein

(76) Inventors: Todd A. Hembrough, Damascus, MD (US); Shawn J. Green, Vienna, VA (US)

Correspondence Address: Kimberly J. Prior, KILPATRICK STOCKTON LLP, 2400 Monarch Tower, 3424 Peachtree Road, N.E., Atlanta, GA, 30326, US

(21) Appl. No.: 09/905,033

(22) Filed: Jul. 12, 2001

Related U.S. Application Data
(60) Provisional application No. 60/217,795, filed on Jul. 12, 2000.

(51) Int. Cl. A61K 38/48; C12N 9/64

(52) U.S. Cl. 424/94.63; 435/226

(57) ABSTRACT

The present invention relates to compositions and uses of nematode anticoagulant proteins (NAPs). More specifically, the invention relates to the use of rNAPc2 and rNAP5 for the inhibition of endothelial cell proliferation and the inhibition of angiogenesis. The invention also relates to methods for the treatment of angiogenesis-mediated diseases, such as cancer.

### CROSS-REFERENCE TO PRIOR RELATED APPLICATIONS

This application claims priority to provisional application Serial No. 60/217,795 filed Jul. 12, 2000.

### FIELD OF THE INVENTION

The present invention relates to compositions and methods for the inhibition of cellular proliferation. More particularly, the present invention relates to the use of a nematode anticoagulant protein to inhibit angiogenesis.

### BACKGROUND OF THE INVENTION

Cellular proliferation is a normal process in all living organisms and is one that involves numerous factors and signals that are delicately balanced to maintain regular cellular cycles. When normal cellular proliferation is disturbed or somehow disrupted, the results can be inconsequential or they can manifest an array of biological disorders such as cancer, abnormal development of embryo, improper formation of the corpus luteum, difficulty in wound healing as well as malfunctioning of inflammatory and immune responses.

Angiogenesis and angiogenesis-mediateddiseases are closely affected by cellular proliferation. As used herein, the term “angiogenesis” means the generation of new blood vessels into a tissue or organ. Under normal physiological conditions, humans or animals undergo angiogenesis only in very specific restricted situations. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane.

Abnormal cellular proliferation leads to the development of an array of biological disorders including cancer, ocular neovascular disase, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, sjogrens, acne, rosacea, phylectenulosis, syphilis, Mycobacteria infections, lipid degeneration, chemical bums, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, rheumatoid arthritis, systemic lupus, polyarteritis, trauma, Wegener's sarcoidosis, scleritis, Stevens-Johnson disease, pemphigoid, radial keratotomy, and corneal graph rejection, rheumatoid arthritis, osteoarthritis, chronic inflammation, ulcerative colitis, Crohn's disease, bartonellosis, atherosclerosis, hemangioma, Osler-Weber-Rendu disease, or hereditary hemorrhagic telangiectasia.

Cancer is the second leading cause of death in the United States, second only to heart disease (which is frequently due in part to atherosclerosis). Since 1990 approximately 12 million new cases of cancer have been diagnosed and five million persons have died of cancer in the United States. Cancer is characterized by abnormal cellular proliferation. Unlike normal cells, cancer cells are atypical in structure and do not have specialized functions. They compete with normal cells for nutrients, eventually killing normal tissue. Cancer cells respond abnormally to control mechanisms that regulate the division of normal cells.

Cancerous, or malignant, tissue can remain localized, invading only neighboring tissue, or can spread to other tissues or organs via the lymphatic system or blood (i.e., metastasize); virtually all tissues and organs are susceptible. It is a disease that is highly unpredictable and has a very high mortality rate. The current treatments for cancer include chemotherapy, surgery and radiation treatments. These radical treatment procedures are highly detrimental to the patient. For example, the typical chemotherapeutic agent is cytotoxic and has a very narrow therapeutic range. Therefore, the chemotherapeutic agent must be carefully administered at a dose that will kill cancer cells but not kill normal cells. These chemotherapeutic agents often leave the patient sick and weakened and are only minimally effective in treating the cancer

Angiogenesis is prominent in solid tumor formation and metastasis. Angiogenic factors have been found associated with several solid tumors such as rhabdomyosarcomas, retinoblastoma, Ewing's sarcoma, neuroblastoma, and osteosarcoma. A tumor cannot grow beyond a certain size without a blood supply to provide nutrients and remove cellular wastes. Tumors in which angiogenesis is important include solid tumors, and benign tumors such as acoustic neuroma, neurofibroma, trachoma and pyogenic granulomas. Prevention of angiogenesis can halt the growth of these tumors and the resultant damage to the animal due to the presence of the tumor.

It should be noted that angiogenesis has been associated with blood-borne tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors.

Angiogenesis is important in two stages of tumor metastasis. The first stage where angiogenesis stimulation is important is in the vascularization of the tumor which allows tumor cells to enter the blood stream and to circulate throughout the body. After the tumor cells have left the primary site, and have settled into the secondary, metastasis site, angiogenesis must occur before the new tumor can grow and expand. Therefore, prevention of angiogenesis could lead to the prevention of metastasis of tumors and possibly contain the neoplastic growth at the primary site.

Knowledge of the role of angiogenesis in the maintenance and metastasis of tumors has led to a prognostic indicator for breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by therapeutic means could possibly lead to cessation of the recurrence of the tumors.

The hypothesis that tumor growth is angiogenesis dependent was first proposed in 1971 by Judah Folkman (N. Engl. Jour. Med. 285:1182 1186, 1971) The hypothesis proposes that once a tumor has started, every increase in tumor cell population must be preceded by an increase in new capillaries converging on the tumor. Therefore, cellular proliferation, particularly endothelial cell proliferation plays a major role in the metastasis of cancer. If this abnormal proliferation activity could be repressed, then the tumor would not grow. Therapies directed at control of the cellular proliferative processes could lead to the abrogation or mitigation of these diseases.

Ocular neovascular disease is also mediated by angiogenesis. This disease is characterized by invasion of new blood vessels into the structures of the eye such as the retina or cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales' disease, Behset's disease, infections causing a retinitis or choroiditis, presumed ocular histoplasmosis, Best's disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogenic-related factors contributes to the destruction of the joint. At a later stage, the angiogenic factors would promote new bone formation. Therapeutic intervention that prevents the bone destruction could halt the progress of the disease and provide relief for persons suffering with arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula.

Angiogenesis is also involved in normal physiological processes such as reproduction and wound healing. Angiogenesis is an important step in ovulation and also in implantation of the blastula after fertilization. Prevention of angiogenesis could be used to induce amenorrhea, to block ovulation or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Tissue factor pathway inhibitor (TFPI) has been shown to have antiproliferative activity on human and other animal endothelial cells. TFPI is a glycoprotein protease inhibitor that plays an important role in the regulation of tissue factor induced blood coagulation. Blood coagulation is a cascade reaction consisting of an intrinsic and extrinsic pathway resulting in the conversion of fibrinogen to fibrin. In the extrinsic pathway, tissue damage releases tissue thromboplastin, or factor III, which combines with Ca2+ ions and factor VIII, plus tissue phospholipids and enzymes from tissue damage, to activate Factor X, the beginning of the common pathway. The intrinsic pathway cascade involves factor XII and platelet phospholipids activating Factor XI, which with Ca2+ activates factor IX, factor IX combines with factor VIII, Ca2+ and platelet phospholipids to activate factor X. Endothelial release of factor XII-platelet aggregation—changes in the platelet structure. Lipids, such as prostacyclins, released from the platelets initiate clotting cascade,—leads to factor X activation, just as in the extrinsic method.

When factor X is activated, the final common pathway is initiated. Factor X, factor V and phospholipids from the cell membranes of damaged tissue and from platelets together form prothrombin activator. Prothrombin activator, and Ca2+, convert prothrombin to thrombin which converts fibrinogen to fibrin, forming the clot.

TFPI prevents the formation of factor VIIa/tissue factor and binds to the active site of factor Xa. The primary sequence indicates that the protein contains three Kunitz-type enzyme inhibitor domains. The first of these domains is required for the inhibition of the VIIa/tissue factor complex. The second Kunitz-type domain inhibits factor Xa. Generally, TFPI is found in plasma, in platelets and on endothelium.

rNAPc2 is the nematode anticoagulant protein which is functionally similar to human tissue factor pathway inhibitor. It is a factor X dependent inhibitor of factor VIIa and regulates the extrinsic pathway, thus mirroring the activity of TFPI in its ability to block factor VIIa proteolytic activity. rNAP5 inhibits the proteolytic activity of factor Xa, mirroring the second activity of TFPI. rNAPc has previously been shown to reduce deep vein thrombosis and is currently being used as an injectable anticoagulant.

Several types of compounds have previously been used to prevent angiogenesis. Taylor et al. have used protamine to inhibit angiogenesis, see Taylor et al., Nature 297:307 (1982). The toxicity of protamine limits its practical use as a therapeutic. Folkman et al. have disclosed the use of heparin and steroids to control angiogenesis. See Folkman et al.,Science 221:719 (1983) and U.S. Pat. Nos. 5,001,116 and 4,994,443. Steroids, such as tetrahydrocortisol, which lack gluco and mineral corticoid activity, have been found to be angiogenic inhibitors.

Other factors found endogenously in animals, such as a 4 kDa glycoprotein from bovine vitreous humor and a cartilage derived factor, have been used to inhibit angiogenesis. Cellular factors such as interferon inhibit angiogenesis. For example, interferon a or human interferon β has been shown to inhibit tumor-induced angiogenesis in mouse dermis stimulated by human neoplastic cells. Interferon β is also a potent inhibitor of angiogenesis induced by allogeneic spleen cells. See Sidky et al., Cancer Research 47:5155-5161 (1987). Human recombinant a interferon (alpha/A) was reported to be successfully used in the treatment of pulmonary hemangiomatosis, an angiogenesis-induced disease. See White et al., New England J Med. 320:1197-1200 (1989).

Other agents which have been used to inhibit angiogenesis include ascorbic acid ethers and related compounds. See Japanese Kokai Tokkyo Koho No. 58-131978. Sulfated polysaccharide DS 4152 also shows angiogenic inhibition. See Japanese Kokai Tokkyo Koho No. 63-119500. A fungal product, fumagillin, is a potent angiostatic agent in vitro. The compound is toxic in vivo, but a synthetic derivative, AGM 12470, has been used in vivo to treat collagen II arthritis. Fumagillin and 0-substituted fumagillin derivatives are disclosed in EPO Publication Nos. 0325199A2 and 0357061A1.

PCT Application No. WO 92/14455 to Kaplan et al. is directed to a method for controlling abnormal concentration of TNF-a by administering thalidomide or thalidomide derivatives to a patient with toxic concentrations of TNF-a.

The above compounds are either topical or injectable therapeutics. Therefore, there are drawbacks to their use as a general angiogenic inhibitor and lack adequate potency. For example, in prevention of excessive wound healing, surgery on internal body organs involves incisions in various structures contained within the body cavities. These wounds are not accessible to local applications of angiogenic inhibitors. Local delivery systems also involve frequent dressings which are impracticable for internal wounds, and increase the risk of infection or damage to delicate granulation tissue for surface wounds.

Thus, a method and composition are needed that are capable of inhibiting angiogenesis and which are easily administered. A simple and efficacious method of treatment would be through the oral route. If an angiogenic inhibitor could be given by an oral route, the many kinds of diseases discussed above, and other angiogenic dependent pathologies, could be treated easily. The optimal dosage could be distributed in a form that the patient could self-administer.

### SUMMARY OF THE INVENTION

The present invention relates generally to compositions that comprise a nematode anticoagulant protein. Preferred nematode anticoagulant proteins of the invention include, but are not limited to, rNAPc2 and rNAP5.

The invention also relates to uses of nematode anticoagulant proteins. The methods include the inhibition of endothelial cell proliferation, the inhibition of angiogenesis, and the treatment of angiogenesis-mediated diseases. Such angiogenesis related diseases include, but are not limited to, cancers; inflammatory conditions, such as Crohn's disease and rheumatoid arthritis; ulcerative diseases, such as ulcerative colitis; ocular diseases, such as corneal neovascular diseases; and immune diseases, such as acquired immune deficiency disease.

Accordingly, it is an object of the present invention t provide a compositions containing a nematode anticoagulant protein.

It is another object of the present invention to provide compositions containing rNAPc2.

It is another object of the present invention to provide compositions containing rNAP5.

It is an object of the present invention to provide methods of inhibiting endothelial cell proliferation in vitro by administering a nematode anticoagulant protein.

It is another object of the present invention to provide methods of inhibiting endothelial cell proliferation in vivo by administering a nematode anticoagulant protein.

It is an object of the present invention to provide methods of inhibiting angiogenesis by administering in vitro a nematode anticoagulant protein.

It is another object of the present invention to provide methods of inhibiting angiogenesis by administering in vivo a nematode anticoagulant protein.

It is yet another object of the present invention to provide methods for inhibiting angiogenesis in a human or animal.

It is a further object of the invention to provide methods for inhibiting angiogenesis in a human or animal have a disease.

It is an object of the present invention to provide methods of treating angiogenesis-mediated diseases.

It is another object of the present invention to provide methods of treating cancer.

It is yet another object of the present invention to provide methods of treating angiogenesis-mediated diseases with minimal side-effects.

These and other objects, features and advantages of the present invention will become apparent after a review of the following detailed description of the disclosed embodiments and the appended claims.

### BRIEF DESCRIPTION OF THE FIGURES

FIG. 1 illustrates the inhibition of LLC primary tumor growth with rNAPc2.

FIG. 2 depicts dose response of LLC primary tumors to rNAPc2.

FIG. 3 shows dose response of LLC experimental metastasis to rNAPc2.

FIG. 4 demonstrates the effect of NAP proteins on LLC metastases.

FIG. 5 depicts the effect of rNAPc2 on B16 melanoma metastasis

FIG. 6 demonstrates that neither rNAPc2 nor rNAP5 inhibit the growth of serum stimulated growth of tumor cell lines.

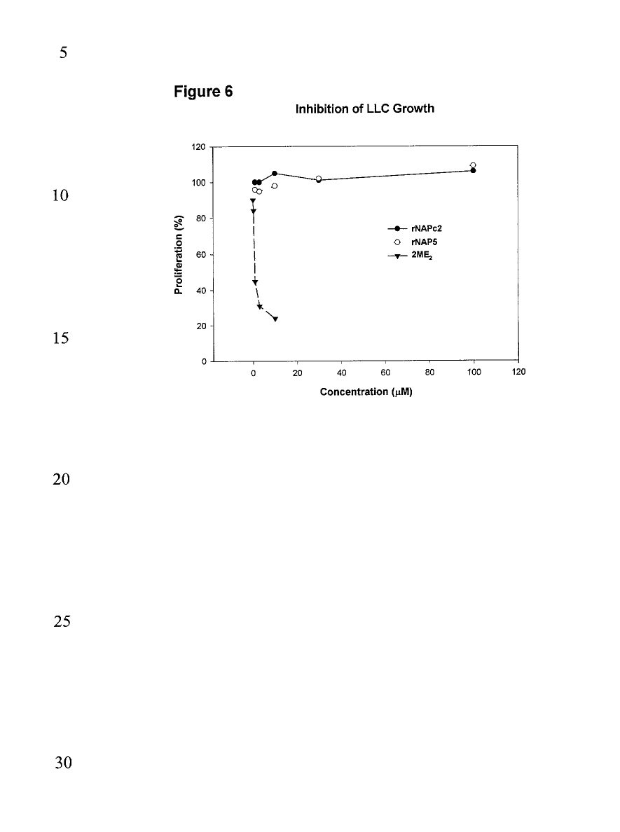

FIG. 7 demonstrates that neither rNAPc2 nor rNAP5 inhibit the growth of bFGF stimulated proliferation of human umbilical vein endothelial cells (HUVECs).

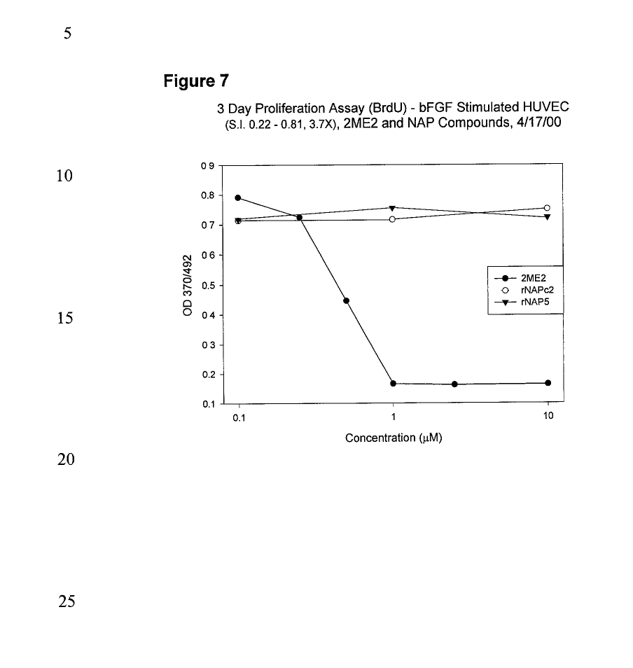

FIG. 8 illustrates the ability of rNAPc2 to inhibit angiogenesis in the Martigel Assay.

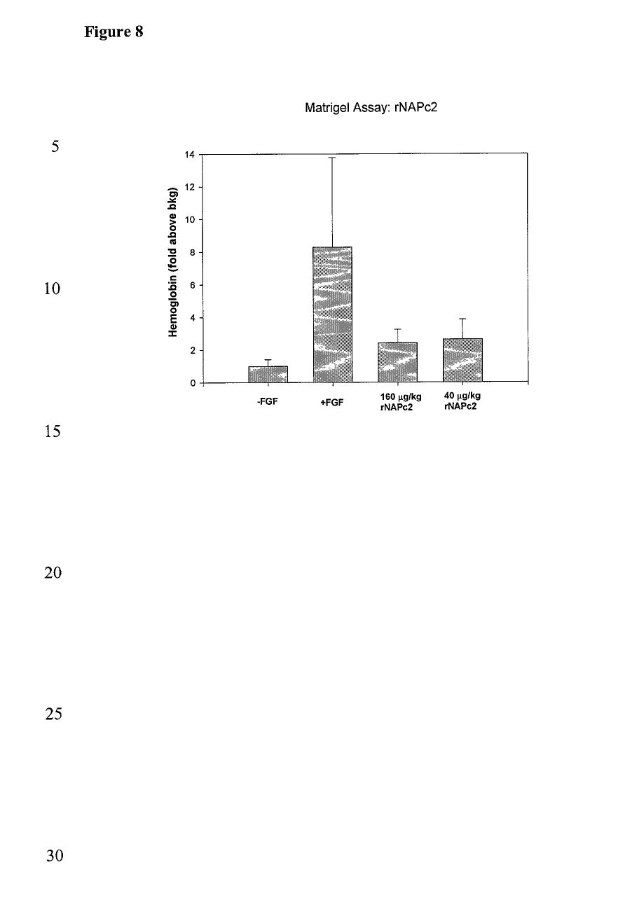

FIG. 9 compares the effect of varying dosages of rNAPc2 on the inhibition of angiogenesis in the Marigel Assay.

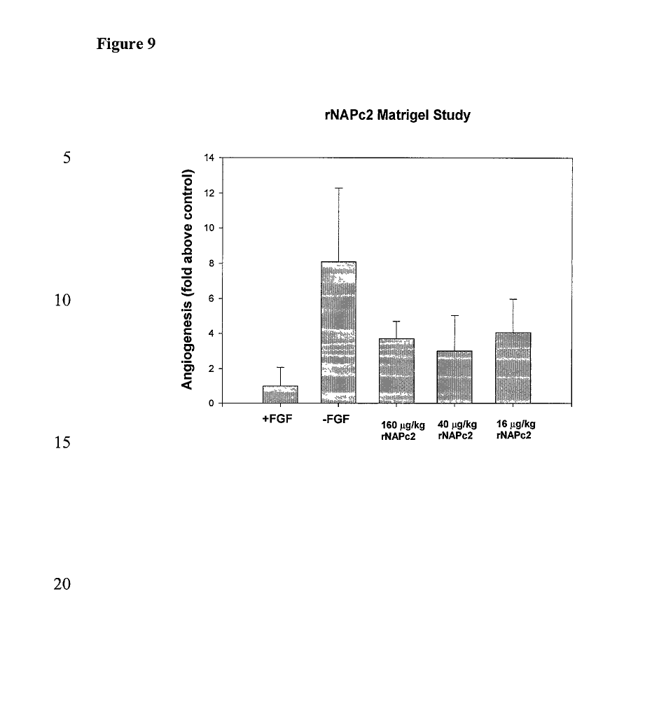

### DETAILED DESCRIPTION OF THE INVENTION

The present invention relates generally to compositions that comprise a nematode anticoagulant protein. The invention also relates to uses of nematode anticoagulant proteins and the compositions containing them. The methods include the inhibition of endothelial cell proliferation, the inhibition of angiogenesis, and the treatment of angiogenesis-mediated diseases.

In one aspect, the present invention comprises a method of inhibiting endothelial cell proliferation with a nematode anticoagulant protein (NAP). Nonlimiting examples of such NAPs include rNAPc2 and rNAP5. Although the invention will be described in terms of these two exemplifies NAPs, it should be understood that the scope of the invention includes the use of any nematode anticoagulant protein. Other NAPs can be used in the present invention in a manner similar to the methods, compositions, and doses described for rNAPc2 and rNAP5.

Tissue factor pathway inhibitor (TFPI) exhibits potent anti-proliferative activity on human and other animal cells, particularly endothelial cells by inhibiting the coagulation cascade. TFPI prevents the formation of factor VIIa/tissue factor and binds to the active site of factor Xa. The primary sequence indicates that the protein contains three Kunitz-type enzyme inhibitor domains. The first of these domains is required for the inhibition of the VIIa/tissue factor complex. The second Kunitz-type domain inhibits factor Xa.

Nematode anticoagulant proteins also affect the coagulation cascade. The protein rNAPc2 is functionally similar to human tissue factor pathway inhibitor. It is a factor X dependent inhibitor of factor VIIa and regulates the extrinsic pathway, thus mirroring the primary activity of TFPI in its ability to block factor VIIa proteolytic activity. The rNAP5 protein inhibits the proteolytic activity of factor Xa, mirroring the second activity of TFPI. Due to the similarities between TFPI and NAPs, the present inventors studied the ability of NAPs to inhibit endothelial cell proliferation and angiogenesis.

Through initial studies, the inventors investigated the ability of NAP proteins to inhibit the proliferation of human umbilical vein endothelial cells (HUVECs) and Lewis lung lymphoma. It was determined that NAPs do not inhibit bFGF stimulated proliferation of HUVECs, or of the serum stimulated growth of tumor cell lines.

TFPI inhibition of endothelial cell proliferation is mediated through binding to the VLDL receptor. However, neither rNAPc2 nor rNAP5 binds to the VLDL receptor. Since TFPI can inhibit both factor VIIa/TF and factor Xa, the antitumor activity of TFPI may be mediated by inhibition of these coagulation factors. The inventors theorized that rNAPc2 may function in a manner similar to TFPI, i.e., by inhibiting the induction of coagulation through fVIIa/TF.

In another aspect, the present invention comprises a method of inhibiting tumor metastatic growth. Metastatic growth is mediated by angiogenesis. Since the major physiological activity of fVIIa/TF complexes is activation of fXa, the inventors sought to determine whether fXa activity was also necessary for primary and metastatic tumor growth and/or for angiogenesis. In order to assess this, rNAP5, a specific inhibitor of fXa, was studied. This protein is closely related to rNAPc2, but has no inhibitory activity toward factor VIIa/TF complexes. The rNAP5 protein was found to modestly inhibit tumor growth in a Lewis lung carcinoma primary tumor model and in the B16 melanoma experimental metastasis model.

Taken together these data implicate the activity of fVIIa/TF in the progression of tumor growth, but show that the major physiological target of this activity, factor Xa, is not required for tumor growth. This suggests the existence of a second, as yet unidentified, target for the tumorigenic activity of the fVIIa/TF complex.

In another aspect the invention comprises a method of inhibiting angiogenesis. To further characterize the mechanism of action of nematode anticoagulant proteins and determine whether or not they inhibit angiogenesis, experimental angiogenesis experiments were performed using the Matrigel plug assay. The results of these experiments suggest that the fVIIa/TF complex plays an important role in the angiogenic model. For example, in the Matrigel assay rNAPc2 was shown to be a very potent inhibitor of angiogenesis. At doses up to 160 ug/kg, there was a significant inhibition of angiogenesis, with maximal inhibition of 78% at 160 ug/kg rNAPc2. Decreasing the amount of rNAPc2 treatment only modestly decreases the antiangiogenic activity of rNAPc2.

The dosage of the compound will depend on the condition being treated, the particular compound, and other clinical factors such as weight and condition of the human or animal and the route of administration of the compound.

The nematode anticoagulant compounds of the present invention can be administered alone or in compositions that contain the compound. Thus, the invention also comprises compositions containing a nematode anticoagulant protein. These compositions include those suitable for oral, rectal, ophthalmic, nasal, topical, vaginal or parenteral, administration. The compositions may conveniently be presented in unit dosage form and may be prepared by conventional pharmaceutical techniques. Such techniques include the step of bringing into association the active ingredient and the physiological carrier(s) or excipient(s). A physiological carrier or excipient is one that does produce toxic side effects when administered in vivo. In general, the compositions are prepared by uniformly and intimately bringing into association the active ingredient with liquid carriers or finely divided solid carriers or both, and then, if necessary, shaping the product.

Compositions of the present invention suitable for oral administration may be presented as discrete units such as capsules, cachets, or tablets, each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus. By predetermined amount, it is meant the amount of nematode anticoagulant protein for a given dose.

A tablet may be made by compression or molding, optionally with one or more additional ingredients. Compressed tablets may be prepared by compressing, in a suitable machine, the active ingredient in a free-flowing form such as a powder or granules, optionally mixed with a binder, lubricant, inert diluent, preservative, or surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the active ingredient therein.

Compositions suitable for topical administration in the mouth include lozenges, including sublingual lozenges, comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Compositions suitable for topical administration to the skin may be presented as ointments, creams, gels and pastes comprising the ingredient to be administered in a physiologically acceptable carrier. A preferred topical delivery system is a transdermal patch containing the ingredient to be administered.

Compositions for rectal administration may be presented as a suppository with a suitable base comprising, for example, cocoa butter or a salicylate.

Compositions suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable compositons, wherein the carrier is a liquid, for example, a nasal spray or as nasal drops, include aqueous or oily solutions of the active ingredient.

Compositions suitable for vaginal administration may be presented as pessaries, tamports, creams, gels, pastes, foams or spray formulations containing in addition to the active ingredient such carriers as are known in the art to be appropriate.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein recited, or an appropriate fraction thereof, of the administered ingredient. An effective dose or angiogenesis-inhibiting dose of the present invention is from about 0.1 ug/kg/day to about 2 mg/kg/day. A preferred dose range is between about 0. 1 ug/kg/day to about 160 ug/kg/day. In one embodiment of the invention an effective dose is from about 1.5 ug/kg/day to about 5.0 ug/kg/day.

It should be understood that in addition to the ingredients, particularly mentioned above, the formulations of the present invention may include other agents conventional in the art having regard to the type of formulation in question, for example, those suitable for oral administration may include flavoring agents.

It is known in the art that certain diseases are mediated by angiogenesis. The phrases “mediated by angiogenesis” or “angiogenesis-mediated disease” mean that angiogenesis is related to at least one component of the disease. Therefore, the compounds of the present invention, nematode anticoagulant proteins, which are antiangiogenic are useful for the treatment of such angiogenesis-mediated diseases. Thus, in yet another aspect, the present invention comprises methods for the treatment of angiogenesis-mediated diseases.

Corneal neovascularization is mediated by angiogenesis. Diseases associated with corneal neovascularization that can be treated according to the present invention include, but are not limited to, diabetic retinopathy, retinopathy of prematurity, corneal graft rejection, neovascular glaucoma, retrolental fibroplasia, epidemic keratoconjunctivitis, Vitamin A deficiency, contact lens overwear, atopic keratitis, superior limbic keratitis, pterygium keratitis sicca, sjogrens, acne, rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, trauma, rheumatoid arthritis, systemic lupus, polyarteritis, Wegeners' sarcoidosis, scleritis, Stevens-Johnson disease, pemphigoid, radial keratotomy, and corneal graph rejection.

Diseases associated with retinal/choroidal neovascularization that can be treated according to the present invention include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcets' disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma, and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

Chronic inflammation is mediated by angiogenesis. Diseases associated with chronic inflammation can be treated by the compositions and methods of the present invention. Diseases with symptoms of chronic inflammation include inflammatory bowel diseases, such as Crohn's disease and ulcerative colitis; psoriasis; sarcoidosis; and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and, thus, maintains the chronic inflammatory state. Inhibition of angiogenesis by the compositions and methods of the present invention would prevent the formation of the granulomas and alleviate the disease.

The compositions and methods of the present invention can be used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis by the compositions and methods of the present invention inhibits the formation of the sprouts and prevents the formation of granulomas.

Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The compositions and methods of the present invention are also capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease is active. The granulomas are created by the angiogenic capillary sprouts that provide a constant supply of inflammatory cells.

The compositions and methods of the present invention can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease which can be treated according to the present invention is rheumatoid arthritis. Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

It is known in the art that tumor growth and the metastasis of cancer are mediated by angiogenesis. Although different cancers have different etiologies and causes, the growth of all tumors require the growth of blood vessels. Metastasis also requires the growth of blood vessels to sustain the cancer growth. Therefore, by inhibiting angiogenesis, the compounds of the present invention are useful for the treatment of cancers generally. For example, the present invention comprises the treatment of hemangiomas, Osler-Weber-Rendu disease, or hereditary hemorrhagic telangiectasia, solid or blood-borne tumors, Kaposi's sarcoma, breast cancer, Ewing's sarcoma, sarcoid, rhabdomyosarcoma, retinblastoma, neuroblastoma, osteosarcoma, leukemia, acoustic neuroma, neurofibroma, and pyogenic granulomas.

Angiogenesis is also associated with many of the ancillary diseases that occur in acquired immune deficiency syndrome. Thus, the compounds of the present invention are useful for the treatment of acquired immune deficiency syndrome.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Lewis Lung Carcinoma Experimental Mestastisis

Groups of five C57BL/6J mice were injected with 5×10⁴ B16BL6 melanoma cells or LLC cells and were subsequently treated intraperitoneally with various concentrations of human TFPI, rNAPc2, rNAP5 or equal volume of diluent control. This treatment was initiated three days after tumor cell inoculation.

Two weeks after the intravenous injection of tumor cells, the mice were sacrificed and necropsied. The lungs were removed and, in the case of B16BL6 metastases, the number of pulmonary colonies was counted under a dissecting microscope. In the case of the LLC metastases, lungs were weighed and compared to untreated controls, and to normal lungs without tumors.

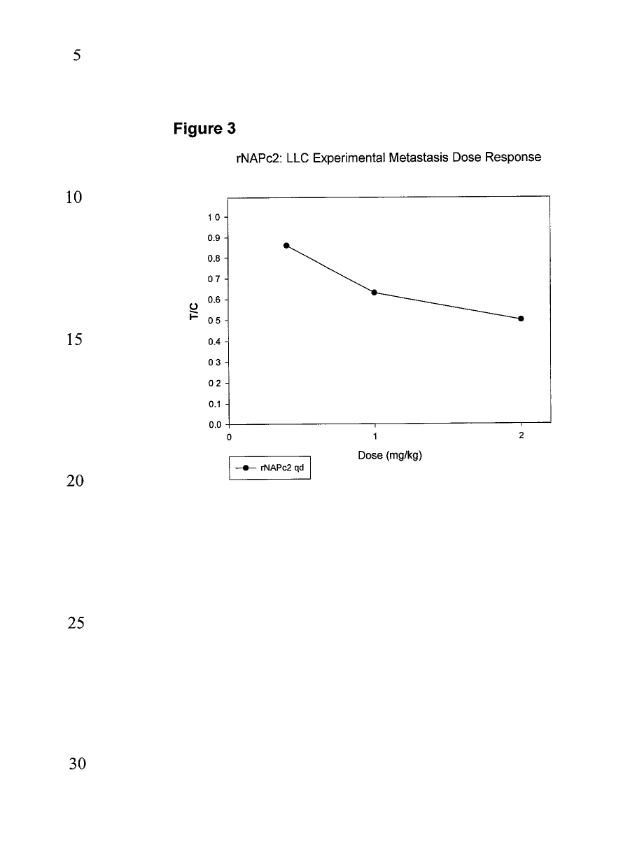

The results of this Experiment are provided in FIG. 3. The results were analyzed for statistical significance using the Student's t-test. As there results illustrate, rNAPc2 exhibits a significant dose dependent inhibition of the growth of lung metastases. As with the primary tumor model, repeated dosing resulted in severe mortality. Deaths were only seen after many daily doses. One animal was found dead after 11, 13, 14, and 16 days of treatment. In each case there was evidence of hemorrhage near the site of peritoneal injection.

### Example 2

### Lewis Lung Carcinoma Metastasis Experiments

Animals were studied using a spontaneous metastatic model. Lewis lung carcinoma (1×10⁶) were injected into the hind footpad of 6-8 week old male C57BL/6J mice (Jackson Laboratories, Bar Harbor, Minn.). Fourteen days later, the primary tumor was resected by amputation of the rear leg just below the head of the femur. Animals with resected tumors were randomly divided into treatment groups and compounds were administered every day beginning day 1 following ligation.

After sacrifice by CO₂ asphyxiation, lungs were removed, weighed, and placed in 10% buffered formalin or Carnoy fixative. Lung weights of treated groups were compared after subtraction of the average lung weights of age-matched normal mice.

LLC metastasis experiments were performed using two models: an EXPERIMENTAL METASTASIS MODEL, and a SPONTANEOUS METASTASIS MODEL (see Example 4). In the EXPERIMENTAL METASTASIS MODEL, tumor cells were injected through the tail vein, and allowed to home to the lungs, where they extravasated into the lung parenchyma. Several papers have shown that the early steps in tumor metastasis are dependent upon coagulation. In the blood, tumor cells form aggregates with platelets and fibrin, and this aggregation enhances cell seeding to the lungs, and extravasation into the tissue. In the absence of fibrinogen, or the presence of coagulation inhibitors, seeding is inefficient, and very few tumors appear in the lungs. Using this model, we sought to assess the effect of rNAPc2 on the growth of metastases. Treatment was initiated on day 3, when tumor cell homing to the lung parenchyma was complete. In this way, the experiment assessed the ability of rNAPc2 to inhibit the growth of the metastases, and did not address any of the earlier steps in tumor metastasis.

### Example 3

### Lewis Lung Carcinoma Primary Tumor Experiments

Groups of five C57BL/6J mice were injected subcutaneously with 2.5×10⁵ B16BL6 melanoma cells and were subsequently treated with various doses of rNAPc2, rNAP5, or cyclophosphamide as a control. The treatment was initiated within a week after tumor cell inoculation, when the primary tumor had an approximate volume of 100 mm³ and was continued every day, or every other day, for 10 to 12 days.

Tumor growth was recorded every other day using calipers to measure the tumor dimensions. Tumor volume was calculated using the following formula: (length)×(width)²×(π/6). The tumor size in the treated animals was compared to the tumor size in the control animals. Statistical analysis was performed using the Student's t-test.

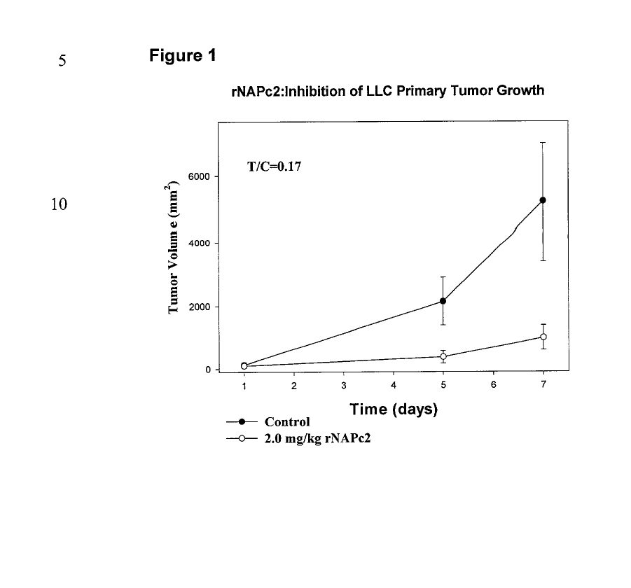

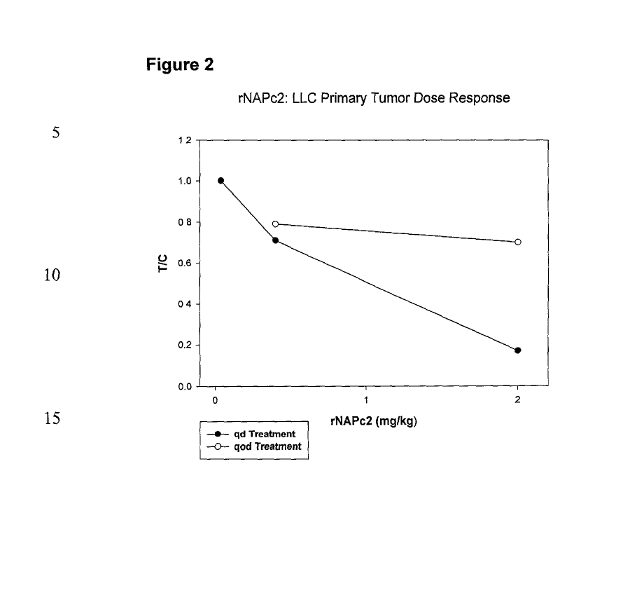

At least 5 experiments were performed using the LLC primary tumor model. The results of these experiments are summarized in Table 1 below and in FIGS. 1 and 2. At the highest dose tested, 2 mg/kg, rNAPc2 induced significant inhibition of tumor growth (FIG. 1 T/C=0.17). However, at this dose significant mortality was observed after 5 days of treatment. Since the half-life of rNAPc2 is >18hr, mortality maybe the result of accumulation of rNAPc2 in the serum levels of rNAPc2 as a consequence of the repeat daily dosing. Consistent with this, when treatments were given every other day, mortality was markedly diminished. However, dosing on alternate days resulted in diminished efficacy.

TABLE 1

| | T/C | Mortality | |
|---|---|---|---|
| 2.0 mg/kg qd | 0.17 | 40% | (2/5) |
| 2.0 mg/kg qod | 0.70 | 0% | (0/5) |
| 0.4 mg/kg qd | 0.71 | 20% | (1/5) |
| 0.4 mg/kg qod | 0.79 | 20% | (1/5) |

### Example 4

### Lewis Lung Carcinoma Spontaneous Metastasis

The second metastasis model assessed was a model for SPONTANEOUS METASTASES. In this experiment, tumor cells were injected into the footpad of a mouse, where they grow as a primary tumor. On approximately day 15 the leg containing the tumor was ligated at the femur, and removed.

Treatment was initiated the following day. In this model, tumor cells metastasize to the lung while the primary tumor grows and, after removal of the primary tumor, the rate of growth of the lung metastases increases significantly. Angiostatin was first identified as a naturally occurring suppressor of metastatic tumor growth in this model.

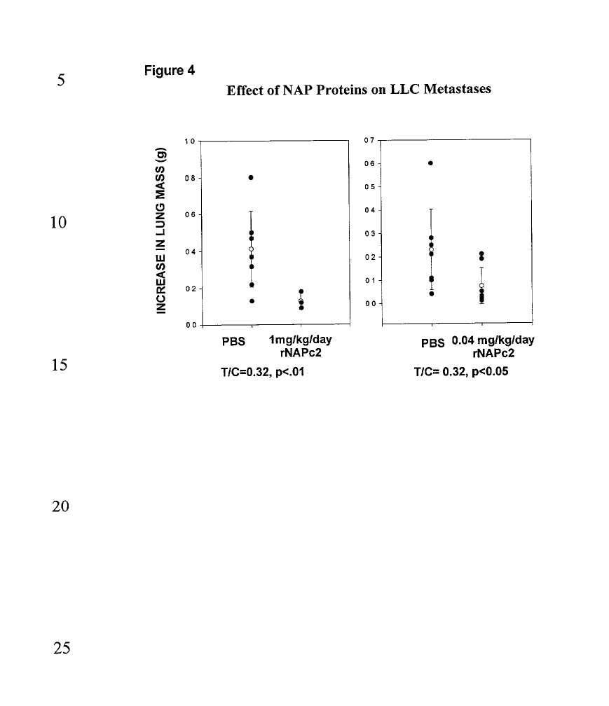

As in the experimental metastatic model, rNAPc2 treatment also targets the growth of the metastases in the lung. In these experiments, we found very good inhibition of metastatic tumor growth by rNAPc2 in the range of 0.04 mg/kg up to 1 mg/kg (FIG. 4). These doses induced 70% inhibition of metastatic growth. The lower dose was not associated with mortality in the treatment group. In an experiment with much higher standard deviation (44%) than those described above (5-10%), a dose of 0.4 mg/kg failed to inhibit the formation of LLC metastases (T/C=0.83±0.36).

### Example 5

### B16 Melanoma Experimental Metastasis

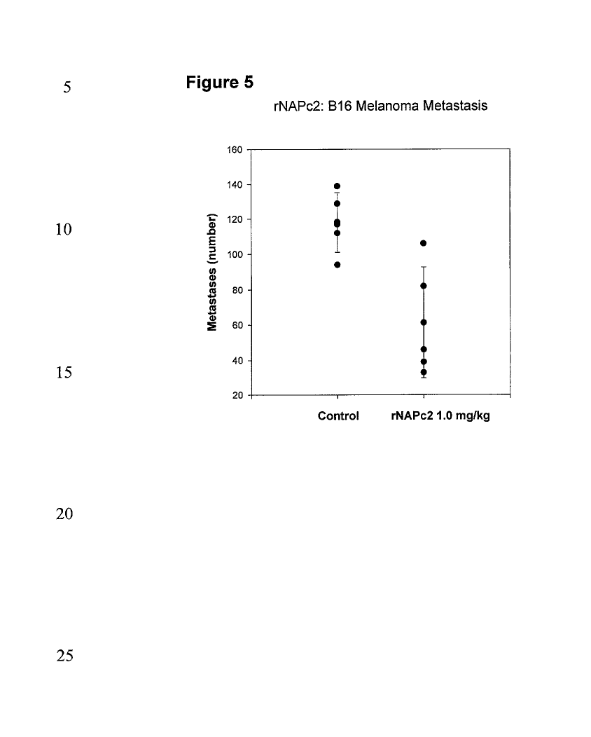

These B16 experiments were performed identically to the LLC experimental metastasis experiments, with one exception. Quantification of tumor load was performed by counting metastatic foci on the surface of the lung, using a microscope. In the B16 model, metastatic foci on the surface of the lungs were counted using a microscope. The results of this experiment are presented in FIG. 5 and show the total number of metastases present after treatment.

In several experiments, we saw modest but statistically significant inhibition of B16 melanoma metastases in rNAPc2 treatment groups. FIG. 5 shows 48% inhibition by treatment with 1.0 mg/kg. This result was confirmed in a second B16 experiment, where the dose of treatment was increased, and the effect of route of treatment was investigated. Treatment with 2.0 mg/kg i.p resulted in about 50% inhibition of lung metastases, while the same dose s.c resulted in only 20% inhibition of B16 metastases.

### Example 6

To further characterize the mechanism of action of rNAPc2, experimental angiogenesis experiments were performed using the Matrigel plug assay. In this assay, 500 ul of Matrigel with bFGF added (2 ug/ml) was injected subcutaneously in the abdomen (Day 1). Treatment was initiated on day 2, and continued through day 8. Plugs were then surgically removed, weighed and processed to measure hemoglobin.

In the Matrigel assay rNAPc2 is a very potent inhibitor of angiogenesis. At doses up to 160 ug/kg, there was a significant inhibition of angiogenesis, with maximal inhibition of 78% at 160 ug/kg rNAPc2. Decreasing the amount of rNAPc2 treatment only modestly decreased the antiangiogenic activity of rNAPc2. At the lowest dose tested (4 ug/kg), angiogenesis was inhibited 57%. When doses higher than 160 ug/kg were tested, there was a significant amount of hemorrhage and clotting in the Matrigel plug, and adjacent tissue. The plugs with clots were not processed. At lower doses of rNAPc2, no clotting or evidence of hemorrhage was seen.

It should be understood, that the foregoing relates only to preferred embodiments of the present invention and that numerous modifications or alterations may be made therein without departing from the spirit and the scope of the invention as set forth in the appended claims

1. A method of inhibiting endothelial cell proliferation, comprising administering to an endothelial cell an effective amount of a nematode anticoagulant protein.

2. The method of claim 1 wherein the nematode anticoagulant protein is rNAPc2.

3. The method of claim 1 wherein the nematode anticoagulant protein is rNAP5.

4. The method of claim 1 wherein the endothelial cell is in culture.

5. The method of claim 1 wherein the endothelial cell is in vivo.

6. The method of claim 5 wherein the endothelial cell is in a human or animal.

7. The method of claim 6 wherein the human or animal has an endothelial cell proliferative mediated disease.

8. The method of claim 7 wherein the disease is selected from the group consisting of diabetic retinopathy, retinopathy of prematurity, corneal graph rejection, neovascular glaucoma, retrolental fibroplasia, epidemic keratoconjunctivitis, Vitamin A deficiency, contact lens overwear, atopic keratitis, superior limbic keratitis, pterygium keratitis sicca, sjogren's syndrome, acne rosacea, phylectenulosis, syphilis, Micobacteria infections other than leprosy, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus erythematosis, polyarteritis, Wegener's sarcoidosis, scleritis, Stevens-Johnson disease, radial keratotomy, macular degeneration, sickle cell anemia, sarcoid, pseudoxanthoma elasticum, Paget's disease, vein occlusion, artery occlusion, carotid obstructive disease, chronic uveitis, chronic vitritis, Lyme's disease, Eales' disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, post-laser complications, rubeosis, abnormal proliferation of fibrovascular or fibrous tissue, proliferative vitreoretinopathy, Bartonellosis, hemangiomas, Osler-Weber-Rendu disease, solid tumors, blood-borne tumors, acquired immune deficiency syndrome, ocular neovascular disease, age-related macular degeneration, osteoarthritis, diseases caused by chronic inflammation, Crohn's disease, ulceritive colitis, tumors of rhabdomyosarcoma, tumors of retinblastoma, tumors of Ewing's sarcoma, tumors of neuroblastoma, tumors of osteosarcoma, leukemia, psoriasis, atherosclerosis, acoustic neuroma, neurofibroma, trachoma, pyogenic granulomas, and pemphigoid.

9. The method of claim 7 wherein the disease is a cancer.

10. The method of claim 9 wherein the cancer is selected from the group consisting of Kaposi's sarcoma, sarcoid, hemangiomas, Osler-Weber-Rendu disease, solid tumors, blood-borne tumors, rhabdomyosarcoma, retinblastoma, Ewing's sarcoma, neuroblastoma, osteosarcoma, leukemia, acoustic neuroma, neurofibroma, pyogenic granulomas, and breast cancer.

11. The method of claim 6 wherein the nematode anticoagulant protein is administered orally, sublingually, buccally, parenterally, subcutaneously, intravenously, intramuscularly, nasally, rectally, vaginally, transdermally, or topically.

12. The method of claim 6 wherein the angiogenesis-inhibiting amount is between approximately 0.1 ug/kg/day and approximately 160.0 ug/kg/day.

13. The method of claim 12 wherein the angiogenesis-inhibiting amount is between approximately 1.5 ug/kg/day and approximately 5.0 ug/kg/day.

14. The method of claim 6 wherein the nematode anticoagulant protein is administered in the form of a tablet, capsule, cachet, lozenge, or suppository.

15. The method of claim 6 wherein the nematode anticoagulant protein is administered in the form of a solution, suspension, or emulsion.

16. The method of claim 6 wherein the nematode anticoagulant protein is administered in the form of a powder, aerosol, spray, pastille, ointment, cream, paste, foam, gel, tamport, or pessary.

17. A method of inhibiting angiogenesis in a human or animal, comprising administering to the human or animal an angiogenesis-inhibiting amount of a nematode anticoagulant protein.

18. The method of claim 17 wherein the nematode anticoagulant protein is rNAPc2.

19. The method of claim 17 wherein the nematode anticoagulant protein is rNAP5.

20. The method of claim 17 wherein the nematode anticoagulant protein is administered orally, sublingually, buccally, parenterally, subcutaneously, intravenously, intramuscularly, nasally, rectally, vaginally, transdermally, or topically.

21. The method of claim 17 wherein the angiogenesis-inhibiting amount is between approximately 0.1 ug/kg/day and approximately 160.0 ug/kg/day.

22. The method of claim 21 wherein the angiogenesis-inhibiting amount is between approximately 1.5 ug/kg/day and approximately 5.0 ug/kg/day.

23. The method of claim 17 wherein the nematode anticoagulant protein is administered in the form of a tablet, capsule, cachet, lozenge, or suppository.

24. The method of claim 17 wherein the nematode anticoagulant protein is administered in the form of a solution, suspension, or emulsion.

25. The method of claim 17 wherein the nematode anticoagulant protein is administered in the form of a powder, aerosol, spray, pastille, ointment, cream, paste, foam, gel, tamport, or pessary.

26. A method of treating an angiogenesis-mediated disease in a human or animal comprising administering to the human or animal an effective amount of a nematode anticoagulant protein.

27. The method of claim 26 wherein the nematode anticoagulant protein is rNAPc2.

28. The method of claim 26 wherein the nematode anticoagulant protein is rNAP5.

29. The method of claim 26 wherein the nematode anticoagulant protein is administered orally, sublingually, buccally, parenterally, subcutaneously, intravenously, intramuscularly, nasally, rectally, vaginally, transdermally, or topically.

30. The method of claim 26 wherein the angiogenesis-inhibiting amount is between approximately 0.1 ug/kg/day and approximately 160.0 ug/kg/day.

31. The method of claim 30 wherein the angiogenesis-inhibiting amount is between approximately 1.5 ug/kg/day and approximately 5.0 ug/kg/day.

32. The method of claim 26 wherein the nematode anticoagulant protein is administered in the form of a tablet, capsule, cachet, lozenge, or suppository.

33. The method of claim 26 wherein the nematode anticoagulant protein is administered in the form of a solution, suspension, or emulsion.

34. The method of claim 26 wherein the nematode anticoagulant protein is administered in the form of a powder, aerosol, spray, pastille, ointment, cream, paste, foam, gel, tamport, or pessary.
